(19) United States
(12) Patent Application Publication
(10) Pub. No.: US 2010/0226863 A1
(43) Pub. Date: Sep. 9, 2010

(54) HOMEOPATHIC MEDICAMENT USEFUL FOR IMMUNOMODULATION

(76) Inventor: Roberto Piraino, Curitiba (BR)

Correspondence Address: HULSEY IP INTELLECTUAL PROPERTY LAWYERS, P.C., 919 Congress Avenue, Suite 919, AUSTIN, TX, 78701, US

(21) Appl. No.: 12/599,199

(22) PCT Filed: Nov. 8, 2007
(86) PCT No.: PCT/BR07/00306

§ 371 Date: May 21, 2010

(30) Foreign Application Priority Data
Mar. 9, 2007 (BR) PI 0702089-9

(51) Int. Cl. A61K 9/12; A61K 33/36

(52) U.S. Cl. 424/45; 424/621

(57) ABSTRACT

Homeopathic immunomodulator medicament, auxiliary in the treatment of the immunolog Formula: formed by 1000 ml of the hydro-alcoholic solution, and variables of Aconitum napellus D 10, Arsenicum album D 19, Asa foetida D 6, Bryonia alba D18, Calcarea carbonica D 20, Ipecacuanha D 19, Pulsatilla nigricans D 18, Ricinus communis D 17, Thuya occidentalis D 19, Lachesis muta D 18. ONCOLOGIC DROPS, INHALER AND INJECTION Formulas: formed by 1000 ml of the hydro-alcoholic solution, and variables of Aconitum napellus D 10. Arsenicum album D 19, Asa foetida D 6, Bryonia alba D 18, Calcarea carbonica D 20, Pulsatilla nigricans D 18, Ricinus communis D 17, Thuya occidentalis D 19, Lachesis muta D 18. BLISTER Formula: Aconitum napellus D 16. Arsenicum album D 21, Asa foetida D 12, Bryonia alba D 9, Calcarea carbonica D 27, Ipecacuanha D 23, Pulsatilla nigricans D 22, Ricinus communis D 19, Thuya occidentalis D 23, Lachesis muta D 20. CREAM, GEL and VAGINAL CAPSULE Formula: Aconitum napellus D 10, Arsenicum album D 19, Asa foetida D 6, Bryonia alba D 18, Calcarea carbonica D 20. Ipecacuanha D 19, Pulsatilla nigricans D 18, Ricinus communis D 17, Thuya occidentalis D 19, Lachesis muta D 18.

Patent Request for the Invention of an immunomodulator homeopathic medicament, auxiliary in the treatment of diseases of the immunological system, highly diluted formulation, does not preset toxicity (absence of genotoxicity in the resultant medicament, in human lymphocytes, stimulated by phytohemagglutinin), prescribed in pathologies where the immunological system (non-restrictive) is compromised.

This homeopathic medicament is developed from components which are listed in the Brazilian Homeopathic Pharmacopoeia and it is presented in six different ways: drops, inhaler, injection, blister, cream and vaginal capsules. The combination and the sequel of dynamization are essential to reach the final product. This medicine induces the organism to use its own resources, mobilizing the natural defenses of the weak body. It is a new way of Immunomodulatory Therapy, directed to specific cells, the macrophages, which contribute to the immunological response. These cells, in the presence of the mentioned medicament, decrease the production and liberation of the cytokine Tumor necrosis factor (TNFα). The clinic-pharmacological approach, different from the conventional allopathic approach, makes this medicament indicated to pathologies where the person is immune-compromised.

The components of this pharmaceutical specialty with homeopathic techniques, called medicament, are:

AIDS drops: Aconitum napellus CH2; Arsenicum album CH5; Asa foetida T.M.; Bryonia alba CH5; Calcarea carbônica CH5; Lachesis muta CH5; Ipecacuanha CH5; Pulsatilla nigricans CH5; Ricinus communis CH5 and Thuya occidentalis CH5.

Cancer drops, inhaler both for cancer and AIDS, and injection: Aconitum napellus CH2; Arsenicum album CH5; Asa foetida T.M.; Bryonia alba CH5; Calcarea carbonica CH5; Lachesis muta CH5; Pulsatilla nigricans CH5; Ricinus communis CH5 and Thuya occidentalis CH5.

Blister: Aconitum napellus CH8; Arsenicum album CH7; Asa foetida CH6; Bryonia Alba CH8; Calcarea carbonica CH12; Lachesis muta CH7, Ipecacuanha CH9; Pulsatilla nigricans CH9; Ricinus communis CH7 and Thuya occidentalis CH9.

Cream, gels and Vaginal capsules: Aconitum napellus CH2; Arsenicum album CH5; Asa foetida T.M.; Bryonia Alba CH5; Calcarea carbônica CH5; Lachesis muta CH5, Ipecacuanha CH5; Pulsatilla nigricans CH5; Ricinus communis CH5 and Thuya occidentalis CH5.

Beyond the acknowledged actions of each component separately, it is undeniable that the combination of these, presents a measurable effect in different experimental models and clinical rehearsals that it is amplified than the action of each component separately, for example: experiences in tumor-bearing mice showed that the tumor stopped growing, which allows us to speculate about the participation of the immunological system in tumor control.

In vitro studies prove that the medicament object of this, does not affect directly tumor cells, or normal lymphocytes, although it acts on macrophages, which in some way stimulates the lymphocytes, and they increase their cytotoxic action in and unspecified way. Experiments using infiltrating tumor lymphocytes allow us to infer that the medicament aforementioned acts as an inductor of endogenous cytokines.

Clinical studies have shown that this homeopathic medicament acts not only on the tumor growth, but it also acts as a controller of inflammatory and infectious diseases, this makes evident that the medicament acts as an immunomodulator in cases where the immunological system is compromised, exercising an stimulating action on the macrophages, and they stimulate lymphocytes increasing their cytotoxic, and even in high concentration it does not present toxicity neither in optical nor in scanning electron microscopy, macrophages present morphology of activated cells, sprawled cells, big nucleus, with non-condensed chromatin and many microvillus and pseudopods. Results showed that the TNF alpha production in vitro decreases after repeated dosages of the medicament in case, suggesting that this is one of the action mechanisms of this medicament.

A thorough exam is requested to check the interrelation between this medicament and the macrophages of diverse diseases. This knowledge can be extended to the selective influence in the state of macrophage activation, what can be of great help in the manipulation of this homeopathic medicament in pathologies of low immunity.

Due to its properties, the immunomodulatory pharmaceutical specialty with homeopathic techniques, the resulting medicament is highly effective to treat patients with cancer, aids, rheumatoid arthritis, hepatitis C, septicemic cases, chronic infectious diseases, acute inflammatory diseases, arthropathies, immunological-based diseases, benign tumor pathology, gastrointestinal lesions, endocrinopathies, genital-urinary infections, gynecological infections, dermatitis and other immune-compromising conditions, being able to stimulate, modulate and accelerate the action of macrophages and lymphocytes.

As previously mentioned, the components of the medicament in case are inserted in the Brazilian Homeopathical Pharmacopoeia, its combination and dynamization sequel are essential to reach the final result of this medicament. The composition and its effects are remarkably positive, acting in the immunological system, activating the healing mechanisms of the organism, not producing side effects in patients that make use of it.

The homeopathic medicament now requested under patent can be employed along with regular/traditional chemotherapy and radiotherapy (allopathic), because it does not interfere in the action of these therapies, neither it presents any kind of side effects, acting as an effective auxiliary and increasing the natural immunological capability of the organism to fight diseases (therefore being highly effective in the 20 oncologic treatments, HIV/AIDS and other previously mentioned diseases where the immune-system is compromised). The medicament can be found in six different pharmaceutical forms: DROPS, INHALER, INJECTION, BLISTER, CREAM and VAGINAL CAPSULES. Before using any of the six different pharmaceutical forms, the package must be vigorously shaken on the palm of the hand.

Efficiency and Safety: The medicament in case is absorbed by the mucous membranes and, due to the absence of lethal dosage; it can be safely used by intravenous injection. The quick absorption makes it appropriate also for inhaling, and no difference is made noticeable in the response from people of different gender, age or race, as also the sublingual drops, which absorption is also very quick, creams and vaginal capsules, without any side effects.

In vivo and in vitro observations confirmed remarkable decrease of the painful systematology of oncologic patients, and improvement in the quality of life, diminish of the tumor and, in many cases, regression of the tumor process, besides the important vitality gain of the patient. It is a safe product, not being related any interaction with other medicaments. It helps the patient to stand the secondary effects of chemotherapy and radiotherapy, as well as the immunological stimulation, making the post-surgical stage safer and briefer. It does not interfere in patients with diabetes, cardiological conditions and can be used safely in convulsive patients and bearers of mental disorders, not having any acknowledgeable intercurrence in the concurrent use of any medicament employed in these treatments, and it does not cause dependence or abuse.

Patients with HIV/AIDS in treatment with anti-retroviral can make use of this medicament concurrently, because clinical studies show the benefits of this use, relieving the side effects of conventional drugs and hindering opportunist diseases, increasing the muscular mass and return to social and professional lives.

Drops Formula for the Treatment of HIV/AIDS—Pharmaceutical specialty with Homeopathic Techniques—HAHNEMANNIAN METHOD: Starting point: Aconitum napellus CH2; Arsenicum album CH5; Asa foetida T.M.; Bryonia Alba CH5; Calcarea carbônica CH5; Lachesis muta CH5, Ipecacuanha CH5; Pulsatilla nigricans CH5; Ricinus communis CH5 and Thuya occidentalis CH5.

Technique First Step:

Preparation 1-A: Calcarea carbônica CH5 100 ml, ethanol 96° GL 230 ml, distilled water 670 ml—use succussion 100 times.

Preparation 2-B: Aconitum napellus CH2 10 100 ml, Asa foetida T.M. 100 ml, ethanol 96° GL 170 ml, distilled water 630 ml—use succussion 100 times.

Preparation 1-C: Preparation 1-A 100 ml, Thuya occidentalis CH5 100 ml, Ipecacuanha CH5 100 ml, Arsenicum album CH5 100 ml, ethanol 96° GL 60 ml, distilled water 540 ml—use succussion 100 times.

Preparation 1-D: Preparation 1-C 100 ml, Bryonia Alba CH5 100 ml, Pulsatilla nigricans CH5 100 ml, Lachesis muta CH5 100 ml, ethanol 96° GL 60 ml, distilled water 540 ml—use succussion 100 times.

Preparation 1-E: Preparation 1-D 100 ml, Ricinus communis CH5 100 ml, ethanol 96° GL 200 ml, distilled water 600 ml—use succussion 100 times.

Preparation 1-F: Preparation 1-E 100 ml, ethanol 96° GL 270 ml, distilled water 630 ml—use succussion 100 times.

Technique Second Step:

First Stage—Preparation 1-F 100 ml, preparation 2-B 100 ml, ethanol 96° GL 240 ml, distilled water 560 ml—use succussion 100 times.

Second Stage—First stage 100 ml, ethanol 96° GL 270 ml, distilled water 630 ml—use succussion 100 times.

Third Stage—second stage 100 ml, ethanol 96° GL 70 ml, distilled water 830 ml—use succussion 100 times.

Technique Third Step:

From the previous third stage, make three (3) dilutions 1.10 in distilled water, using succussion 100 times after each dilution, and after quality control (innocuousness, pyrogen and sterility) the product for sublingual use is obtained to be administered.

Conservation: amber-colored glass container, well-closed, protected from heat and direct light. Expiration date: six (6) months.

Final Formula Drops HIV/AIDS: Aconitum napellus D 10, Arsenicum album D 19, Asa foetida D 6, Bryonia alba D 18, Calcarea carbônica D 20, Ipecacuanha D 19, Pulsatilla nigricans D 18, Ricinus communis D 17, Thuya occidentalis D 20, Lachesis muta D 18.

Drops Formula for Oncologic Treatment—Pharmaceutical Specialty with Homeopathic Techniques—HAHNEMANNIAN METHOD: Starting point: Aconitum napellus CH2; Arsenicum album CH5; Asa foetida T.M.; Bryonia 25 Alba CH5; Calcarea carbônica CH5; Lachesis muta CH5, Pulsatilla nigricans CH5; Ricinus communis CH5 and Thuya occidentalis CH5.

Technique First Step:

Preparation 1-A: Calcarea carbônica CH5 100 ml, ethanol 96° GL 230 ml, distilled water 670 ml—use succussion 100 times.

Preparation 2-B: Aconitum napellus CH2 100 ml, Asa foetida T.M. 100 ml, ethanol 96° GL 170 ml, distilled water 630 ml—use succussion 100 times.

Preparation 1-C: Preparation 1-A 100 ml, Thuya occidentalis CH5 100 ml, Arsenicum album CH5 100 ml, ethanol 96° GL 130 ml, distilled water 570 ml—use succussion 100 times.

Preparation 1-D: Preparation 1-C 100 ml, Bryonia alba CH5 100 ml, Pulsatilla nigricans CH5 100 ml, Lachesis muta CH5 100 ml, ethanol 96° GL 60 ml, distilled water 540 ml—use succussion 100 times.

Preparation 1-E: Preparation 1-D 100 ml, Ricinus communis CH5 100 ml, ethanol 96° GL 200 ml, distilled water 600 ml—use succussion 100 times.

Preparation 1-F: Preparation 1-E 100 ml, ethanol 96° GL 270 ml, distilled water 630 ml—use succussion 100 times.

Technique Second Step:

First Stage—Preparation 1-F 100 ml, preparation 2-B 100 ml, ethanol 96° GL 240 ml, distilled water 560 ml—use succussion 100 times.

Second Stage—First stage 100 ml, ethanol 96° GL 270 ml, distilled water 630 ml—use succussion 100 times.

Third Stage—Second stage 100 ml, ethanol 96° GL 70 ml, distilled water 830 ml—use succussion 100 times.

Technique Third Step:

From the previous third stage, make three (3) dilutions 1.10 in distilled water, using succussion 100 times after each dilution, and after quality control (innocuousness, pyrogen and sterility) the product for sublingual use is obtained to be administered.

Conservation: amber-colored glass container, well-closed, protected from heat and direct light. Expiration date: six (6) months.

Final Formula for Oncologic Drops: Aconitum napellus D 10, Arsenicum album D 19, Asa foetida D 6, Bryonia alba D 18, Calcarea carbônica D 20, Pulsatilla nigricans D18, Ricinus communis D 17, Thuya occidentalis D 19, Lachesis muta D 18.

Inhaler Formula—Pharmaceutical Specialty with Homeopathic Techniques: HAHNEMANNIAN METHOD: Starting point: Aconitum napellus CH2, Arsenicum album CH5, Asa foetida T.M., Bryonia Alba CH5, Calcarea carbônica CH5, Lachesis muta CH5, Pulsatilla nigricans CH5, Ricinus communis CH5, Thuya occidentalis CH5.

Technique First Step: Preparation 1-A: Calcarea carbônica CH5 100 ml, ethanol 96° GL 230 ml, distilled water 670 ml—use succussion 100 times.

Preparation 2-B: Aconitum napellus CH2 100 ml, Asa foetida T.M. 100 ml, ethanol 96° GL 170 ml, distilled water 630 ml—use succussion 100 times.

Preparation 1-C: Preparation 1-A 100 ml, Thuya occidentalis CH5 100 ml, Arsenicum album CH5 100 ml, ethanol 96° GL 130 ml, distilled water 570 ml—use succussion 100 times.

Preparation 1-D: Preparation 1-D 100 ml, Bryonia Alba CH5 100 ml, Pulsatilla nigricans CH5 100 ml, Lachesis muta CH5 100 ml, ethanol 96° GL 60 ml, distilled water 540 ml—use succussion 100 times.

Preparation 1-E: Preparation 1-D 100 ml, Ricinus communis CH5 100 ml, ethanol 96° GL 200 ml, distilled water 600 ml—use succussion 100 times.

Preparation 1-F: Preparation 1-E 100 ml, ethanol 96° GL 270 ml, distilled water 630 ml—use succussion 100 times.

Technique Second Step:

First Stage—Preparation 1-F 100 ml, preparation 2-B 100 ml, ethanol 96° GL 240 ml, distilled water 560 ml—use succussion 100 times.

Second Stage—First stage 100 ml, ethanol 96° GL 270 ml, distilled water 630 ml—use succussion 100 times.

Third Stage—Second stage 100 ml, ethanol 96° GL 70 ml, distilled water 830 ml—use succussion 100 times.

Technique Third Step:

From the previous third stage, make three (3) dilutions 1.10 in distilled water, using succussion 100 times after each dilution, and after quality control (innocuousness, pyrogen and sterility) the product for inhaler use is obtained to be administered.

Conservation: amber-colored glass container, well-closed, protected from heat and direct light. Expiration date: six (6) months.

Final Inhaler Formula: Aconitum napellus D 10, Arsenicum album D 19, Asa foetida D 6, Bryonia alba D18, Calcarea carbônica D 20, Lachesis muta D 18, Pulsatilla nigricans D 18, Ricinus communis D 17, Thuya occidentalis D 19.

Injection Formula: Pharmaceutical Specialty with Homeopathic Techniques: HAHNEMANNIAN METHOD: Starting point: Aconitum napellus CH2, Arsenicum album CH5, Asa foetida T.M., Bryonia alba CH5, Calcarea carbônica 10 CH5, Lachesis muta CH5, Pulsatilla nigricans CH5, Ricinus communis CH5, Thuya occidentalis CH5.

Technique First Step:

Preparation 1-A: Calcarea carbônica CH5 100 ml, ethanol 96° GL 230 ml, distilled water 670 ml—use succussion 100 times.

Preparation 2-B: Aconitum napellus CH2 100 ml, Asa foetida T.M. 100 ml, ethanol 96° GL 170 ml, distilled water 630 ml—use succussion 100 times.

Preparation 1-C: Preparation 1-A 100 ml, Thuya occidentalis CH5 100 ml, Arsenicum album CH5 100 ml, ethanol 96° GL 130 ml, distilled water 570 ml—use succussion 100 times.

Preparation 1-D: Preparation 1-C 100 ml, Bryonia alba CH5 100 ml, Pulsatilla nigricans CH5 100 ml, Lachesis muta CH5 100 ml, ethanol 96° GL 60 ml, distilled water 540 ml—use succussion 100 times.

Preparation 1-E: Preparation 1-D 100 ml, Ricinus communis CH5 100 ml, ethanol 96° GL 200 ml, distilled water 600 ml—use succussion 100 times.

Preparation 1-F: Preparation 1-E 100 ml, ethanol 96° GL 270 ml, distilled water 630 ml—use succussion 100 times.

Technique Second Step:

First Stage—Preparation 1-F 100 ml, preparation 2-B 100 ml, ethanol 96° GL 240 ml, distilled water 560 ml—use succussion 100 times.

Second Stage—First stage 100 ml, ethanol 96° GL 70 ml, distilled water 830 ml—use succussion 100 times.

Third Stage—Second stage 100 ml, ethanol 96° GL 70 ml, distilled water 830 ml—use succussion 100 times.

Technique Third Step:

From the previous third stage, make three (3) dilutions 1.10 in specific water for injection use, using succussion 100 times after each dilution, then filtering through sterilizing membrane for bottling, and after quality control (innocuousness, pyrogen and sterility) the product for injection use is obtained to be administered.

Conservation: amber-colored glass container, well-closed, protected from heat and direct light. Expiration date: six (6) months.

Final Injection Formula: Aconitum napellus D 10, Arsenicum album D 19, Asa foetida D 06, Bryonia alba D 18, Calcarea carbônica D 20, Lachesis muta D 18, Pulsatilla nigricans D 18, Ricinus communis D 17, Thuya occidentalis D 19.

Blister Formula: Pharmaceutical Specialty with Homeopathic Techniques: HAHNEMANNIAN METHOD: Starting point: Aconitum napellus CH8; Arsenicum album CH7; Asa foetida CH6; Bryonia Alba CH8; Calcarea carbônica CH12; Lachesis muta CH7, Ipecacuanha CH9; Pulsatilla nigricans CH9; Ricinus communis CH7 and Thuya occidentalis CH19.

Technique First Step:

Preparation 1-A: Calcarea carbônica CH12 100 ml, ethanol 96° GL 230 ml, distilled water 670 ml—use succussion 100 times.

Preparation 2-B: Aconitum napellus CH8 100 ml, Asa foetida CH6 100 ml, ethanol 96° GL 170 ml, distilled water 630 ml—use succussion 100 times.

Preparation 1-C: Preparation 1-A 100 ml, Thuya occidentalis CH9 100 ml, Ipecacuanha CH9 100 ml, Arsenicum album CH7 100 Ml, ethanol 96° GL 60 ml, distilled water 540 ml—use succussion 100 times.

Preparation 1-D: Preparation 1-C 100 ml, Bryonia Alba CH8 100 ml, Pulsatilla nigricans CH9 100 ml, Lachesis muta CH7 100 ml, ethanol 96° GL 60 ml, distilled water 540 ml—use succussion 100 times.

Preparation 1-E: Preparation 1-D 100 ml, Ricinus communis CH7 100 ml, ethanol 96° GL 200 ml, distilled water 600 ml—use succussion 100 times.

Preparation 1-F: Preparation 1-E 100 ml, ethanol 96° GL 270 ml, distilled water 630 ml—use succussion 100 times.

Technique Second Step:

First Stage —Preparation 1-F 100 ml, preparation 2-B 100 ml, ethanol 96° GL 240 ml, distilled water 560 ml—use succussion 100 times.

Second Stage—First stage 100 ml, ethanol 96° GL 270 ml, distilled water 630 ml—use succussion 100 times.

Third Stage—Second stage 100 ml, ethanol 96° GL 70 ml, distilled water 630 ml—use succussion 100 times.

Technique Third Step:

From the previous third stage, make three (3) dilutions 1.10 in distilled water, using succussion 100 times after each dilution, and after quality control (innocuousness, pyrogen and sterility) the product for sublingual use is obtained to be administered.

Conservation: amber-colored glass container, well-closed, protected from heat and direct light. Expiration date: six (6) months.

Final Blister Formula: Aconitum napellus D 16, Arsenicum album D 21, Asa foetida D 12, Bryonia alba D 9, Calcarea carbônica D 27, Ipecacuanha D 23, Pulsatilla nigricans D 22, Ricinus communis D 19, Thuya occidentalis D 23, Lachesis muta D 20.

The blister medicament has more medical potency, making possible an increased immunological response when used associated to the other two formulations, which were: drops and inhaler.

Cream Gel and Vaginal Capsules Formula—Pharmaceutical Specialty with Homeopathic Techniques: HAHNEMANNIAN METHOD: Starting point: Aconitum napellus CH2; Arsenicum album CH5; Asa foetida T.M.; Bryonia Alba CH5; Calcarea carbônica CH5; Lachesis muta CH5, Ipecacuanha CH5; Pulsatilla nigricans CH5; Ricinus communis CH5 e Thuya occidentalis CH5.

Technique First Step:

Preparation 1-A: Calcarea carbônica CH5 100 ml, ethanol 96° GL 230 ml, distilled water 670 ml—use succussion 100 times.

Preparation 2-B: Aconitum napellus CH2 100 ml, Asa foetida T.M. 100 ml, ethanol 96° GL 170 ml, distilled water 630 ml—use succussion 100 times.

Preparation 1-C: Preparation 1-A 100 ml, Thuya occidentalis CH5 100 ml, Ipecacuanha CH5 100 ml, Arsenicum album CH5 100 ml, ethanol 96° GL 60 ml, distilled water 540 ml—use succussion 100 times.

Preparation 1-D: Preparation 1-C 100 ml, Bryonia Alba CH5 100 ml, Pulsatilla nigricans CH5 100 ml, Lachesis muta CH5 100 ml, ethanol 96° GL 60 ml, distilled water 540 ml—use succussion 100 times.

Preparation 1-E: Preparation 1-D 100 ml, Ricinus communis CH5 100 ml, ethanol 96° GL 200 ml, distilled water 600 ml—use succussion 100 times.

Preparation 1-F: Preparation 1-E 100 ml, ethanol 96° GL 270 ml, distilled water 630 ml—use succussion 100 times.

Technique Second Step:

First Stage—Preparation 1-F 100 ml, preparation 2-B 100 ml, ethanol 96° GL 240 ml, distilled water 560 ml—use succussion 100 times.

Second Stage—First stage 100 ml, ethanol 96° GL 270 ml, distilled water 630 ml—use succussion 100 times.

Third Stage—Second stage 100 ml, ethanol 96° GL 70 ml, distilled water 830 ml—use succussion 100 times.

Technique Third Step:

From the previous third stage, make three (3) dilutions 1.10 in distilled water, using succussion 100 times after each dilution, and after quality control (innocuousness, pyrogen and sterility) the product for the production of vaginal cream is obtained. For the preparation of other creams, gels and vaginal capsules it is used 10% of the aforementioned product, in proportion to the amount of creams, gels and vaginal capsules manufactured.

Conservation: Creams and Gels—100 grams closed tubes, protected from heat. Expiration date: one (1) year.

Conservation: Vaginal capsules—six (6) units closed blister, protected from heat. Expiration date: one (1) year.

Final Formula: Creams, Gels and Vaginal Capsules—Aconitum napellus D 10, Arsenicum album D 19, Asa foetida D 6, Bryonia alba D 18, Calcarea carbônica D 20, Ipecacuanha D 19, Pulsatilla nigricans D 18, Ricinus communis D 17, Thuya occidentalis D 19, Lachesis muta D 18.

1) “HOMEOPATHIC IMMUNOMODULATOR MEDICAMENT, AUXILIARY IN THE TREATMENT OF THE IMMUNOLOGICAL SYSTEM DISEASES”, DROPS FORMULA FOR HIV/AIDS: Characterized by the fact that, in technique 1^{st} step: Preparation 1-A: Calcarea carbônica CH5 100 ml, ethanol 96° GL 230 ml, distilled water 670 ml, use succussion 100 times, in Preparation 2-B: Aconitum napellus CH2 100 ml, Asa foetida T.M. 100 ml, ethanol 96° GL 170 ml, distilled water 630 ml, use succussion 100 times and in Preparation 1-C: Preparation 1-A 100 ml, Thuya occidentalis CH5 10 100 ml, Ipecacuanha CH5 100 ml, Arsenicum album CH5 100 ml, ethanol 96° GL 60 ml, distilled water 540 ml, use succussion 100 times, in Preparation 1-D: Preparation 1-C 100 ml, Bryonia alba CH5 100 ml, Pulsatilla nigricans CH5 100 ml, Lachesis muta CH5 100 ml, ethanol 96° GL 60 ml, distilled water 540 ml, use succussion as the previous, in Preparation 1-E: Preparation 1-D 100 ml, Ricinus communis CH5 100 ml, ethanol 96° GL 200 ml, distilled water 600 ml, use succussion as the previous, in Preparation 1-F: Preparation 1-E 100 ml, ethanol 96° GL 270 ml, distilled water 630 ml, use succussion as the previous, in Technique 2^{nd} step, First stage: Preparation 1-F 100 ml, Preparation 2-B 100 ml, ethanol 96° GL 240 ml, distilled water 560 ml, use succussion as the previous, in Second stage: First stage 100 ml, ethanol 96° GL 270 ml, distilled water 630 ml, use succussion as the previous, in the Third stage: Second stage 100 ml, ethanol 96° GL 70 ml, distilled water 830 ml, use succussion as the previous, in Technique 3^{rd} step, from the previous third stage make three (3) dilutions in distilled water, using succussion 100 times after each dilution and after quality control (innocuousness, pyrogen and sterility), the product for sublingual use to be administered is obtained.

2) “HOMEOPATHIC IMMUNOMODULATOR MEDICAMENT, AUXILIARY IN THE TREATMENT OF THE IMMUNOLOGICAL SYSTEM DISEASES”, DROPS FORMULA FOR ONCOLOGIC TREATMENT: Characterized by the fact that, in technique 1^{st} step: Preparation 1-A: Calcarea carbônica CH5 100 ml, ethanol 96° GL 230 ml, distilled water 670 ml—use succussion 100 times, in Preparation 2-B: Aconitum napellus CH2 100 ml, Asa foetida T.M. 100 ml, ethanol 96° GL 170 ml, distilled water 630 ml—use succussion 100 times, in Preparation 1-G: Preparation 1-A 100 ml, Thuya occidentalis CH5 100 ml, Arsenicum album CH5 100 ml, ethanol 96° GL 130 ml, distilled water 570 ml—use succussion 100 times, in Preparation 1-D: Preparation 1-C 100 ml, Bryonia Alba CH5 100 ml, Pulsatilla nigricans CH5 100 ml, Lachesis muta CH5 100 ml, ethanol 96° GL 60 ml, distilled water 540 ml—use succussion 100 times, in Preparation 1-E: Preparation 1-D 100 ml, Ricinus communis CH5 100 ml, ethanol 96° GL 200 ml, distilled water 600 ml—use succussion 100 times, in Preparation 1-F: Preparation 1-E 100 ml, ethanol 96° GL 270 ml, distilled water 630 ml—use succussion 100 times, in Technique 2^{d} step, First stage: Preparation 1-F 100 ml, Preparation 2-B 100 ml, ethanol 96° GL 240 ml, distilled water 560 ml—use succussion 100 times, in the Second stage: First stage 100 ml, ethanol 96° GL 270 ml, distilled water 630 ml—use succussion 100 times, in the Third stage: Second stage 100 ml, ethanol 96° GL 70 ml, distilled water 830 ml—use succussion 100 times, in Technique 3^{rd} step: from the previous third stage, make three (3) dilutions 1.10 in distilled water, using succussion 100 times after each dilution, and after quality control (innocuousness, pyrogen and sterility) the product for sublingual use is obtained to be administered.

3) “HOMEOPATHIC IMMUNOMODULATOR MEDICAMENT, AUXILIARY IN THE TREATMENT OF THE IMMUNOLOGICAL SYSTEM DISEASES”, INHALER FORMULA: Characterized by the fact that, in technique 1^{st} step: Preparation 1-A: Calcarea carbônica CH5 100 ml, ethanol 96° GL 230 ml, distilled water 670 ml, use succussion 100 times, in Preparation 2-B: Aconitum napellus CH2 100 ml, Asa foetida T.M. 100 ml, ethanol 96° GL 170 ml, distilled water 630 ml, use succussion 100 times, in Preparation 1-C: Preparation 1-A 100 ml, Thuya occidentalis CH5 100 ml, Arsenicum album CH5 100 ml, ethanol 96° GL 130 ml, distilled water 570 ml, use succussion as the previous, in Preparation 1-D: Preparation 1-C 100 ml, Bryonia Alba CH5 100 ml, Pulsatilla nigricans CH5 100 ml, Lachesis muta CH5 100 ml, ethanol 96° GL 60 ml, distilled water 540 ml, use succussion as the previous, in Preparation 1-E: Preparation 1-D100 ml, Ricinus communis CH5 100 ml, ethanol 96° GL 200 ml, distilled water 600 ml, use succussion as the previous, in Preparation 1-F: Preparation 1-E 100 ml, ethanol 96° GL 270 ml, distilled water 630 ml, use succussion as the previous, in the Technique 2^{nd} step, First stage, Preparation 1-F 100 ml, Preparation 2-B 100 ml, ethanol 96° GL 240 ml, distilled water 560 ml, use succussion as the previous, in the Second stage: First stage 100 ml, ethanol 96° GL 270 ml, distilled water 630 ml, use succussion as the previous, in the Third stage: Second stage 100 ml, ethanol 96° GL 70 ml, distilled water 830 ml, use succussion as the previous, in Technique 3^{rd} step: from the previous third stage, make three (3) dilutions 1.10 in distilled water, using succussion 100 times after each dilution, and after quality control (innocuousness, pyrogen and sterility) the product for inhaler use is obtained to be administered.

4) “HOMEOPATHIC IMMUNOMODULATOR MEDICAMENT, AUXILIARY IN THE TREATMENT OF THE IMMUNOLOGICAL SYSTEM DISEASES” INJECTION FORMULA: Characterized by the fact that, in technique 1^{st} step: Preparation 1-A, Calcarea carbônica CH5 100 ml, ethanol 96° GL 230 ml, distilled water 670 ml, use succussion 100 times, in Preparation 2-B: Aconitum napellus CH2 100 ml, Asa foetida T.M. 100 ml, ethanol 96° GL 170 ml, distilled water 630 ml, use succussion as the previous, in Preparation 1-C: Preparation 1-A 100 ml, Thuya occidentalis CH5 100 ml, Arsenicum album CH5 100 ml, ethanol 96° GL 130 ml, distilled water 570 ml, use succussion as the previous, in Preparation 1-D: Preparation 1-C 100 ml, Bryonia alba CH5 100 ml, Pulsatilla nigricans CH5 100 ml, Lachesis muta CH5 100 ml, ethanol 96° GL 60 ml, distilled water 540 ml, use succussion as the previous, in Preparation 1-E: Preparation 1-D 100 ml, Ricinus communis CH5 100 ml, ethanol 96° GL 200 ml, distilled water 600 ml, use succussion as the previous, in Preparation 1-F: Preparation 1-E 100 ml, ethanol 96° GL 270 ml, distilled water 630 ml, use succussion as the previous, in the Technique 2^{nd} step, First stage, Preparation 1-F 100 ml, Preparation 2-B 100 ml, ethanol 96° GL 240 ml, distilled water 560 ml, use succussion as the previous, in the Second stage: First stage 100 ml, ethanol 96° GL 70 ml, distilled water 830 ml, use succussion as the previous, in the Third stage: Second stage 100 ml, ethanol 96° GL 70 ml, distilled water 830 ml, use succussion as the previous, in Technique 3^{rd} step: from the previous third stage, make three (3) dilutions 1.10 in specific water for injection use, using succussion 100 times after each dilution, then filtering through sterilizing membrane for bottling, and after quality control (innocuousness, pyrogen and sterility) the product for injection use is obtained to be administered.

5) “HOMEOPATHIC IMMUNOMODULATOR MEDICAMENT, AUXILIARY IN THE TREATMENT OF THE IMMUNOLOGICAL SYSTEM DISEASES” BLISTER FORMULA: Characterized by the fact that, in technique 1^{st} step: Preparation 1-A: Calcarea carbônica CH12 100 ml, ethanol 96° GL 230 ml, distilled water 670 ml—use succussion 100 times, in Preparation 2-B: Aconitum napellus CH8 100 ml, Asa foetida CH6 100 ml, ethanol 96° GL 170 ml, distilled water 630 ml—use succussion 100 times, in Preparation 1-C: Preparation 1-A 100 ml, Thuya occidentalis CH9 100 ml, Ipecacuanha CH9 100 ml, Arsenicum album CH7 100 ml, ethanol 96° GL 60 ml, distilled water 540 ml—use succussion 100 times, in Preparation 1-D: Preparation 1-C 100 ml, Bryonia alba CH8 100 ml, Pulsatilla nigricans CH9 100 ml, Lachesis muta CH7 100 ml, ethanol 96° GL 60 ml, distilled water 540 ml—use succussion 100 times, in Preparation 1-E: Preparation 1-D 100 ml, Ricinus communis CH7 100 ml, ethanol 96° GL 200 ml, distilled water 600 ml—use succussion 100 times, in Preparation 1-F: Preparation 1-E 100 ml, ethanol 96° GL 270 ml, distilled water 630 ml—use succussion 100 times, in Technique 2^{nd} step, First stage—Preparation 1-F 100 ml, Preparation 2-B 100 ml, ethanol 96° GL 240 ml, distilled water 560 ml—use succussion 100 times, in the Second stage: First stage 100 ml, ethanol 96° GL 270 ml, distilled water 630 ml—use succussion 100 times, in the Third stage: Second stage 100 ml, ethanol 96° GL 70 ml, distilled water 830 ml—use succussion 100 times, in Technique 3^{rd} step: From the previous third stage, make three (3) dilutions 1.10 in distilled water, using succussion 100 times after each dilution, and after quality control (innocuousness, pyrogen and sterility) the product for sublingual use is obtained to be administered. The blister medicament has more medical potency, making possible an increased immunological response when used associated to the other two formulations, which were: drops and inhaler.

6) “HOMEOPATHIC IMMUNOMODULATOR MEDICAMENT, AUXILIARY IN THE TREATMENT OF THE IMMUNOLOGICAL SYSTEM DISEASES” CREAMS, GELS AND VAGINAL CAPSULES FORMULA—Characterized by the fact that, in technique 1^{st} step: Preparation 1-A: Calcarea carbônica CH5 100 ml, ethanol 96° GL 230 ml, distilled water 670 ml—use succussion 100 times, in Preparation 2-B: Aconitum napellus CH2 100 ml, Asa foetida T.M. 100 ml, ethanol 96° GL 170 ml, distilled water 630 ml—use succussion 100 times, in Preparation 1-C: Preparation 1-A 100 ml, Thuya occidentalis CH5 100 ml, Ipecacuanha CH5 100 ml, Arsenicum album CH5 100 ml, ethanol 96° GL 60 ml, distilled water 540 ml—use succussion 100 times, in Preparation 1-D: Preparation 1-C 100 ml, Bryonia Alba CH5 100 ml, Pulsatilla nigricans CH5 100 ml, Lachesis muta CH5 100 ml, ethanol 96° GL 60 ml, distilled water 540 ml—use succussion 100 times, in Preparation 1-E: Preparation 1-D 100 ml, Ricinus communis CH5 100 ml, ethanol 96° GL 200 ml, distilled water 600 ml—use succussion 100 times, in Preparation 1-F: Preparation 1-E 100 ml, ethanol 96° GL 270 ml, distilled water 630 ml—use succussion 100 times, in the Technique 2^{nd} step, First stage—Preparation 1-F 100 ml, Preparation 2-B 100 ml, ethanol 96° GL 240 ml, distilled water 560 ml—use succussion 100 times, in the Second stage: First stage 100 ml, ethanol 96° GL 270 ml, distilled water 630 ml—use succussion 100 times, in the Third stage: Second stage 100 ml, ethanol 96° GL 70 ml, distilled water 830 ml—use succussion 100 times, in the Technique 3^{rd} step: From the previous third stage, make three (3) dilutions 1.10 in distilled water, using succussion 100 times after each dilution, and after quality control (innocuousness, pyrogen and sterility) the product for the production of vaginal cream is obtained, and for the preparation of other creams, gels and vaginal capsules it is used 10% of the aforementioned product, in proportion to the amount of creams, gels and vaginal capsules manufactured.
